(19) United States
(12) Patent Application Publication
(10) Pub. No.: US 2022/0249361 A1
(43) Pub. Date: Aug. 11, 2022

(54) COSMETIC COMPOSITION COMPRISING A BIFIDOBACTERIUM SPECIES LYSATE, AN EXTRACT OF YEAST OF THE SACCHAROMYCES GENUS, AND A MONOSACCHARIDE AND COSMETIC USES THEREOF

(71) Applicant: L'OREAL, Paris (FR)

(72) Inventors: Marine PEGEON, Chevilly Larue (FR); Gabriel Ahmad KHODR, Chevilly Larue (FR)

(21) Appl. No.: 17/617,498

(22) PCT Filed: Jun. 22, 2020
(86) PCT No.: PCT/EP2020/067353

§ 371 Date: Dec. 8, 2021

(30) Foreign Application Priority Data
Jun. 24, 2019 (FR) FR1906837

(51) Int. Cl. A61K 8/99; A61K 8/9728; A61K 8/60; A61Q 19/00; A61Q 19/08; A61K 8/73

(52) U.S. Cl. CPC A61K 8/99; A61K 8/9728; A61K 8/60; A61K 2800/522; A61Q 19/08; A61K 8/73; A61Q 19/00

(57) ABSTRACT

The present invention relates to a cosmetic composition comprising, in a physiologically acceptable medium: —at least one microorganism of the Bifidobacterium species genus and/or a fraction thereof and/or a metabolite thereof, and —at least one extract of yeast of the Saccharomyces genus, and —at least one monosaccharide chosen from mannose, rhamnose and mixtures thereof, said microorganism being used in the form of a lysate. The present invention also relates to a cosmetic treatment process comprising the application of said composition to the skin; and also the cosmetic uses thereof.

The present invention relates to a cosmetic composition comprising, in a physiologically acceptable medium, a Bifidobacterium species lysate, an extract of yeast of the Saccharomyces genus, and a monosaccharide chosen from mannose, rhamnose, and mixtures thereof. It also relates to its uses and cosmetic treatment processes.

Human skin is constituted of two compartments, namely a deep compartment, the dermis, and a superficial compartment, the epidermis.

The skin also represents a complex ecosystem on which several types of microorganisms, such as bacteria and fungi, proliferate. These microorganisms constitute the skin flora, also called skin microbial flora.

To date, more than 500 bacterial species have been detected on healthy skin with more than 2 million genes. Around 10⁶ bacteria inhabit each cm² of skin. The microbiome of the skin of healthy subjects has been described as exhibiting specificity according to 3 types of zones: wet, dry and oily.

Among the bacterial species detected on the skin, mention may be made of Cutibacterium acnes (also called Propionibacterium acnes) which is one of the bacteria most commonly isolated from human skin.

This species is part of the commensal beneficial resident flora constituted of microorganisms conventionally proliferating on healthy skin, continuously by drawing their nutrients from the skin, and bringing known benefits to the skin.

Indeed, it has been discovered that the abundance of Cutibacterium acnes on the skin was reduced in older women compared to young women Aging-related changes in the diversity of women's skin microbiomes associated with oral bacteria Scientific Reports volume 7, Article number: 10567 (2017); Shift in skin microbiota of Western European women across ageing, Journal of Applied Microbiology, volume 125, issue 3 (2018)).

Furthermore, Cutibacterium acnes, by virtue of the secretion of a RoxP (radical oxygenase of Propionibacterium acnes) enzyme is also known to have antioxidant properties (A novel enzyme with antioxidant capacity produced by the ubiquitous skin colonizer Propionibacterium acnes, Scientific Reports volume 6, Article number: 36412 (2016); Common skin bacteria protect their host from oxidative stress through secreted antioxidant RoxP, Scientific Reports volume 9, Article number: 3596 (2019)).

Thus, Cutibacterium acnes therefore proves to be a target of interest for identifying new active agents that are of use for skin care, in particular for the prevention and/or treatment of the signs of skin ageing and also for the protection of the skin against oxidative stress.

There is therefore a need to provide new active ingredients making it possible to promote, in particular specifically, the growth of Cutibacterium acnes.

Unexpectedly, the inventors have discovered that the combination of a Bifidobacterium species lysate, an extract of yeast of the Saccharomyces genus, and a monosaccharide chosen from mannose and/or rhamnose makes it possible to promote a growth of Cutibacterium acnes greater than that observed with each of said compounds alone, and could be of interest for skin care, in particular for the prevention and/or treatment of the signs of skin ageing and also for the protection of the skin against oxidative stress. This growth of Cutibacterium acnes being in particular specific.

Anti-ageing cosmetic compositions comprising a Bifidobacterium species lysate combined with an extract of yeast of the Saccharomyces genus are available on the market for cosmetic products. However, these compositions do not comprise a monosaccharide chosen from mannose and/or rhamnose as claimed in the context of the present invention. According to Example No. 1 below, mannose alone only promotes moderate growth of Cutibacterium acnes.

However, its addition to the combination of a Bifidobacterium species lysate and an extract of yeast of the Saccharomyces genus makes it possible to obtain, surprisingly, a strong increase in the growth of Cutibacterium acnes.

Thus, a first subject of the present invention is a cosmetic composition comprising, in a physiologically acceptable medium:
at least one microorganism of the Bifidobacterium species genus and/or a fraction thereof and/or a metabolite thereof, and
at least one extract of yeast of the Saccharomyces genus, and
at least one monosaccharide chosen from mannose, rhamnose, and mixtures thereof,

said microorganism being used in the form of a lysate.

The present invention also relates to a cosmetic treatment process for skin care comprising the application to the skin of the composition according to the invention.

In particular, the cosmetic treatment process according to the invention makes it possible to prevent and/or treat the signs of skin ageing, in particular induced by oxidative stress.

Also, the cosmetic treatment process according to the invention makes it possible to protect the skin against oxidative stress.

The present invention also relates to the cosmetic use of the composition according to the invention for preventing and/or treating the signs of skin ageing, in particular induced by oxidative stress.

A subject of the invention is also the cosmetic use of the composition according to the invention for protecting the skin against oxidative stress.

### Definitions

The expression “composition comprising a physiologically acceptable medium” is intended to mean a composition comprising a medium compatible with the skin.

According to one particular embodiment, the pH of the cosmetic composition is between 4 and 7.5, notably between 4.5 and 7, and in particular between 4.7 and 6.5.

The term “skin” is intended to mean all the skin of the human body, and preferably the skin of the face, the skin of the scalp, neckline, neck, arms and forearms, or even more preferably the skin of the face, notably of the forehead, nose, cheeks, chin and area around the eyes.

According to the invention, the term “preventing” or “prevention” is intended to mean reducing the risk of occurrence or slowing down the occurrence of a given phenomenon.

The term “treating” or “treatment” is intended to mean any action that aims to improve the comfort or the well-being of an individual; this term therefore covers both attenuating or relieving and curing.

### DETAILED DESCRIPTION OF THE INVENTION

### Lysate of Microorganism of the Bifidobacterium Genus

As previously specified, the microorganisms of the Bifidobacterium species genus used in the composition according to the invention are used in the form of a lysate.

A lysate commonly denotes a material obtained after the destruction or dissolution of biological cells by a phenomenon termed cell lysis, thus causing the release of the intracellular biological constituents naturally contained in the cells of the microorganism in question.

For the purposes of the present invention, the term “lysate” is used without preference to denote the entire lysate obtained by lysis of the microorganism in question, or only a fraction thereof.

The lysate used is thus totally or partially formed from the intracellular biological constituents and constituents of the cell walls and membranes.

More specifically, it contains the cellular cytoplasmic fraction containing enzymes such as lactic acid dehydrogenase, phosphatases, phosphoketolases or transaldolases and the metabolites. For illustrative purposes, the constituents of the cell walls are in particular peptidoglycan, murein or mucopeptide and teichoic acid, and the constituents of the cell membranes are composed of glycerophospholipids.

This cell lysis may be accomplished by various techniques, for instance osmotic shock, heat shock, by ultrasound, or alternatively under a mechanical stress, for instance centrifugation.

More particularly, this lysate may be obtained according to the technology described in patent U.S. Pat. No. 4,464,362 and especially according to the following protocol.

The microorganism in question of the Bifidobacterium species type is cultured anaerobically in a suitable culture medium, for example according to the conditions described in documents U.S. Pat. No. 4,464,362 and EP 43 128. When the stationary phase of development is reached, the culture medium can be inactivated by pasteurization, for example at a temperature of 60 to 65° C. for 30 min. The microorganisms are then collected via a conventional separation technique, for example membrane filtration or centrifugation, and are resuspended in a sterile physiological NaCl solution.

The lysate may be obtained by ultrasonic disintegration of such a medium so as to release the cytoplasmic fractions, the cell wall fragments and the products derived from metabolism. Next, all the components in their natural distribution are then stabilized in a weakly acidic aqueous solution.

A concentration of about from 0.1 to 50%, in particular from 1 to 20% and especially about 5% by weight of active material(s) relative to the total weight of the lysate is thus generally obtained.

The lysate may be used in various forms: in the form of a solution or in a pulverulent form.

The microorganism belonging to the Bifidobacterium species genus is more particularly chosen from the following species: Bifidobacterium longum Bifidobacterium bifidum, Bifidobacterium breve, Bifidobacterium animalis, Bifidobacterium lactis, Bifidobacterium infantis, Bifidobacterium adolescentis or Bifidobacterium pseudocatenulatum and mixtures thereof.

The species Bifidobacterium longum is most particularly suitable for use in the invention.

For the purposes of the invention, the term “metabolite” denotes any substance resulting from the metabolism of the microorganisms in question according to the invention and also endowed with efficacy for the treatment of the signs of skin ageing and/or for the protection of the skin against oxidative stress.

For the purposes of the invention, the term “fraction” more particularly denotes a fragment of said microorganism endowed with efficacy for the treatment of the signs of skin ageing and/or for the protection of the skin against oxidative stress, by analogy to said whole microorganism.

The product sold under the name Repair Complex CLR® by the company K. Richter GmbH, and which is formed from an inactivated lysate of the species Bifidobacterium longum, is included in the context of the invention.

The active agent forming the lysate belonging to the Bifidobacterium species genus may be formulated in a proportion of at least 0.0001% (expressed as dry weight), in particular in a proportion of from 0.001% to 20% and more particularly in a proportion of from 0.001% to 2% by weight relative to the total weight of the support or of the composition containing it.

Extract of Yeast of the Saccharomyces Genus

The yeasts can be prepared by culture in a conventional yeast culture medium (yeast extract: 10 g/l, pepsin-digested peptone: 7 g/l, glucose: 20 g/l, water: qs).

An aqueous yeast extract, that is to say a yeast extract which, subject to unavoidable losses according to good manufacturing practice, contains all the water-soluble constituents of yeast after the lysis of the yeast and removal by filtration of their membrane debris, will preferentially be chosen. This aqueous yeast extract will preferentially be redissolved.

Preferably, the aqueous extract of yeast of the Saccharomyces genus according to the invention will be prepared by solubilizing in water (preferentially distilled water) whole yeasts of the Saccharomyces, genus by subjecting the suspension thus obtained to protein hydrolysis, by separating the soluble and insoluble phases from the solution obtained following this hydrolysis and by subjecting the soluble phase, recovered at the end of the previous step, to sterilization.

In one preferential embodiment of the invention, the aqueous yeast extract thus obtained can then be optionally dried and provided in powder form. The extract, preferably in powder form, can also be placed in solution (reference will then be made to a yeast extract in solution), in particular an aqueous-alcoholic solution and preferentially an aqueous-glycolic solution (for example a solution constituted of a mixture water and pentylene glycol). In this solution, the aqueous yeast extract will preferentially be added at a concentration between 0.5 and 8%, preferentially between 2 and 7%, even more preferentially between 3 and 5%.

The yeast extract used in the present invention will ultimately comprise at least 50%, preferentially at least 60%, even more preferentially at least 70% or at least 80% water relative to the total weight thereof.

Advantageously, the yeast extract in solution, that can be used according to the present invention, will have the following characteristics:
it will have a pH between 5 and 8, preferentially between 6 and 7,
it will comprise between 20 and 60 g of sugars per litre of solution (g/l), preferentially between 20 and 50 g/l and even more preferentially between 25 and 35 g/l, and/or
it will have a solids concentration of between 10 and 60 g/l, preferentially of between 20 and 50 g/land even more preferentially of between 37 and 47 g/l.

The population of whole yeasts used to prepare this aqueous yeast extract, that is to say before hydrolysis and/or sterilization, will preferentially be from 10^{E}5 to 10^{E}10 colony-forming units per millilitre (or cfu/ml) of aqueous yeast solution.

The aqueous yeast extract will preferentially have the following characteristics:
it will have a total nitrogen content (according to the Kjeldahl method) of from 5 to 15%, preferentially from 6 to 12%, even more preferentially from 7 to 10%, and/or
it will have a content of total free amino acids (according to Sörensen's method) of from 2 to 10%, preferentially from 3 to 7%, even more preferentially from 4 to 6%, and/or
it will have an assimilable amino nitrogen/total nitrogen ratio of 0.4 to 0.7%, preferentially 0.4 to 0.6%, even more preferentially 0.5 to 0.6%.

The protein hydrolysis will preferentially be carried out by chemical or acid hydrolysis or by the use of natural yeast enzymes and it will preferentially be carried out for at least 60%, preferentially at least 80%, even more preferentially at least 90% of all the proteins present in the yeast solution after the yeast lysis.

The separation of the soluble and insoluble phases obtained following the hydrolysis of the proteins will be carry out by any means known to those skilled in the art depending on the nature of the extract sought. This phase separation can for example be carried out by filtration, decanting or centrifugation, filtration being the preferential means. Following this separation of the soluble and insoluble phases, the soluble phase is recovered.

The sterilization may be carried out by any means known to those skilled in the art and in particular by sterilizing filtration. The latter will preferentially be carried out by the use of a membrane filter which the pore size is chosen as a function of the size of the membrane elements that it is desired to eliminate. This technique is well known to those skilled in the art.

Following this sterilization step, the aqueous yeast extract obtained can be dried to provide it in powder form. This drying can also be carried out by any means known to those skilled in the art, for example by evaporation, lyophilization or spray-drying.

In the Saccharomyces genus, mention may be made of the following species: Saccharomyces bailii, Saccharomyces carlsbergensis, Saccharomyces uvarum, Saccharomyces cerevisiae, Saccharomyces delbrueckii, Saccharomyces exiguus, Saccharomyces fermentati, Saccharomyces florentinus, Saccharomyces fragilis Saccharomyces fructuum, Saccharomyces heterogenicus, Saccharomyces oleaginosus, Saccharomyces rosei, Saccharomyces steineri, Saccharomyces boulardii, Saccharomyces kefir, Saccharomyces kluyveri.

Preferentially, a yeast of the species Saccharomyces cerevisiae will be chosen.

Preferentially, this extract does not comprise live yeast.

An example of extract according to the invention is sold by the company Silab (BP 213, 19108 Brive Cedex France) under the name Firmalift® GR. It is in the form of a yellow-coloured, slightly opalescent solution containing 32.0 g/l of sugars. The CAS number for this extract is 8013-01-2. Its EINECS/ELINCS number is 232-387-9. This extract is at a concentration of 4.2% in an aqueous-glycolic solution comprising 7.5% of pentylene glycol and 88.3% of water.

Preferentially, the yeast extract according to the invention will be used at a concentration of from 0.001% to 5%, preferentially from 0.01% to 1% and even more preferentially from 0.02% to 0.5% relative to the weight total of the composition containing it.

Monosaccharide

The composition according to the invention comprises a monosaccharide chosen from mannose, rhamnose, mixtures thereof.

The monosaccharides according to the invention are in the D or L form of mannose and/or rhamnose or their mixture, each form itself being able to be the alpha and/or beta anomer. The preferred forms according to the invention are D-mannose or L-rhamnose.

Mannose is a monosaccharide (simple non-hydrolyzable sugar) constituted of 6 carbons, it is a hexose. Its empirical formula is C₆H₁₂O₆, the same as glucose, of which it is the C₂ epimer (that is to say that its spatial configuration is strictly the same, except for the substituent of carbon 2, where it is reversed compared to glucose).

The monosaccharide in question according to the invention corresponds to formula (I) below.

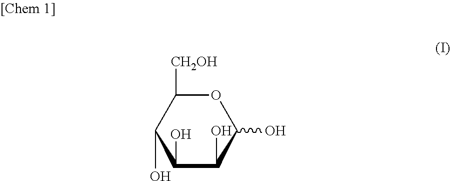

Of course, all of the enantiomeric forms of mannose are considered according to the invention.

The mannose may also be in solvated form (including hydrates) and in the form of a mixture of D and L stereoisomers: DL-mannose.

According to one preferred embodiment, the monosaccharide is the D-mannose of formula (II) below.

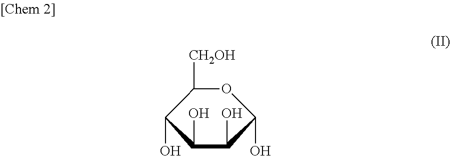

D-Mannose is naturally present in plants, in particular certain fruits, including cranberries, or in hardwood (beech and birch).

As an illustration of D-mannose suitable for the invention, mention may in particular be made of D-mannose sold by the company Danisco Sweeteners® or else the company Symrise®.

Rhamnose (or 6 deoxy mannose) formally constitutes the product of deoxygenation of mannose in C6. Rhamnose is found in nature in the L form. L-rhamnose, are marketed, for example, by the company Danisco Sweeteners® as well as the company Symrise.

In general, the monosaccharide, preferably mannose, more preferably D-mannose, can be used from 0.01% to 20% by weight, preferably from 0.05% to 10% by weight, and most preferentially from 0.1% to 5% by weight, relative to the total weight of the composition containing it.

Oligosaccharide, Polysaccharide

The composition according to the invention may also comprise at least one oligosaccharide and/or polysaccharide in the group constituted of inulin, a fructooligosaccharide, a glucooligosaccharide, oligosaccharides derived from soya beans, pyrodextrins, an isomaltooligosaccharide, a xylooligosaccharide, a transgalactooligosaccharide and mixtures thereof.

Oligosaccharides and polysaccharides are carbohydrates.

The oligosaccharides and/or polysaccharides can be produced in particular from glucose, galactose, xylose, maltose, sucrose, lactose, starch, xylan, hemicellulose, inulin, gums, in particular from acacia gum, or a mixture thereof.

The term “oligosaccharides” is intended to mean oligoholosides or alternatively oligosides, which are oligomers made up of a number n of monosaccharides via one or more alpha or beta glycosidic bonds, the number n being between 2 and 10, preferably between 2 and 6. Mention may in particular be made of fructooligosaccharides, glucooligosaccharides, oligosaccharides derived from soya, pyrodextrins, isomaltooligosaccharides, xylooligosaccharides and transgalactooligosaccharides.

The term “polysaccharide” is intended to mean polyosides, polyholosides, or else complex carbohydrates which are polymers constituted of several units n of monosaccharides linked together by saccharide bonds, the number n being greater than or equal to 11, preferably between 11 and 200, even better still between 11 and 100, even more preferably between 20 and 80. Mention may particular be made of inulin.

By way of example, mention will be made of the following oligosaccharides and/or polysaccharides.

Inulin

Inulin is particularly abundant in the rhizomes of plants, in particular the Jerusalem artichoke and chicory from which it is extracted industrially. It is also found in other plants belonging to the family Asteraceae such as Jerusalem artichokes or dahlia onions and burdock. It is considered a soluble dietary fibre.

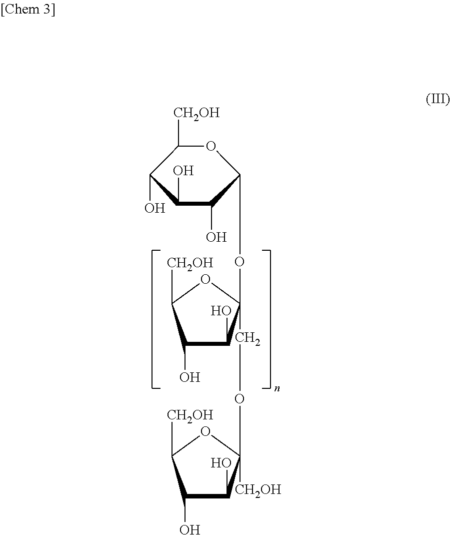

Inulins are polydispersed linear polymers of general formula (III): GFn (G=glucose, F=Fructose, n ranging from 2 to more than 60), the fructose units being linked together by a β (2→1) bond. Inulins therefore correspond to a chain of fructose units terminated by a glucose unit.

Among the inulins which can be used and which are commercially available, mention may be made of Inutec H25P, n of between 2 and 7, and Inutec N25 from Orafti (average n=25).

Fructooligosaccharides

Fructooligosaccharides (or FOSs), also called oligofructoses or oligofructans, are short chains of fructose linked together by β-1,2 bonds.

The fructooligosaccharides (or FOSs) correspond to general formula (IV): G(F)ₙ where G is a glucose unit, F is fructose unit and n ranges from 1 to 10.

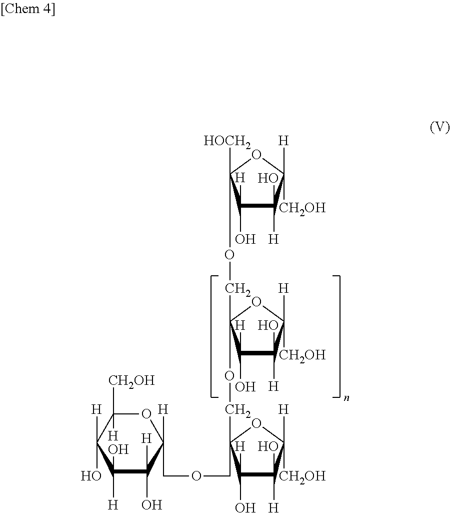

Fructooligosaccharides (FOSs) are produced:
by partial enzymatic hydrolysis of inulin (ex: Raftilose® from Orafti-Belgique), or
by enzymatic synthesis from sucrose (ex: Actilight® from Beghin Meiiji industries-France), or
by extraction from yacon or jicama (Polymnia sonchifolia, synonym: Smallanthus sonchifolia); in particular, the extraction is carried out in the absence of solvent, by cold pressing of yacon tubers.

The FOS used in the invention can be a mixture of FOSs. Mention may in particular be made of the GF2+GF3+GF4 mixtures such as Quantom FOS95 from Quantum hi-Tech or Actilight® from Beghin Meiiji industries-France, the latter corresponding to a mixture of 37% GF2, 53% GF3 and 10% GF4.

Glucooligosaccharides GOSs

Glucooligosaccharides (GOSs), or oligoglucans, are oligosaccharides constituted of a sequence of α-1,6-linked glucose units which can also contain α-1,2; α-1,3; α-1,4 bonds. They are synthesized by a transglucosylation reaction catalyzed by enzymes of the glucan-sucrase family.

Preferably, the glucooligosaccharides are oligosaccharides constituted of a sequence of α-1,6- and α-1,2-linked glucose units.

Advantageously, the number of glucose units is between 2 and 10, even better still between 4 and 6, even more preferably the number of glucose units is 4.

In addition, the glucooligosaccharides can be synthesized by the polymerization of glucose molecules, a reaction catalyzed by a specific enzyme of the glycosyltransferase type, extracted and purified from a bacterial strain of Leuconostoc mesenteroides. This reaction requires the use of an acceptor: maltose (Glucose-Glucose) but also of a glucose donor: sucrose (Glucose-Fructose).

In a preferred embodiment, the glucooligosaccharides (GOSs) have the formula (V) below:

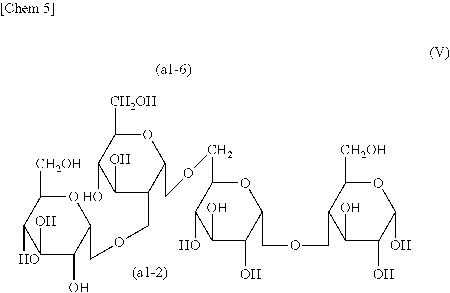

Among the GOSs which can be used and which are commercially available, mention may be made of Bioecolia® from Solabia.

Soya-Derived Oligosaccharides

They are extracted directly from the soya bean and do not require any enzymatic treatment. They naturally contain raffinose and stachyose, the formula of which is [α-D-Gal-(1→6)-]ₘ-α-D-Glu-(1→2)-β-D-Fru with m=1 for raffinose and m=2 for stachyose.

Among the oligosaccharides which can be used, mention may be made of Soya-oligo from Calpis Food Ind. Japan.

Pyrodextrins

Pyrodextrins are a mixture of oligosaccharides from the hydrolysis of starch.

Isomaltooligosaccharide

They are produced from starch. These are α-(1,6)-linked glucose oligomers with a degree of polymerization of between 2 and 5. By way of example, Isomalto900P from Showa Sango may be used.

Xylooligosaccharide

Xylooligosaccharides are oligosaccharides constituted of β-(1,4)-linked xylose. By way of example, Xylo 95P from Suntory limited may be used.

Transgalactooligosaccharide

They are linear oligomers of galactose, of chemical structure α-D-Glucose-(1→4)-[β-D-Galactose-(1→6)-]n (2<n<5) obtained by fermentation of lactose. By way of example, TOS 100 from Yakult Honsha Co. Ltd Japan may be used.

The oligosaccharide(s) and/or polysaccharide(s) according to the invention may be present in the composition according to the invention (i.e. the total concentration of oligosaccharide(s) and/or polysaccharide(s) in the composition according to the invention) in a concentration of between 0.01% and 20% by weight, preferably between 0.05% and 10% by weight, even better still between 0.05% and 5% by weight relative to the total weight of the composition.

Mixture of Oligosaccharides and/or Polysaccharides

According to one preferred embodiment of the invention, the oligosaccharide(s) and/or polysaccharide(s) are used in the composition according to the invention in the form of a mixture.

In a first embodiment of the invention, it may be a mixture of oligosaccharides of the same type. For example, as mentioned above, a mixture of FOSs may be used, in particular a mixture of GF2+GF3+GF4, such as Quantom FOS95 from Quantum hi-Tech or Actilight® from Beghin Meiiji industries-France, the latter corresponding to a mixture of 37% GF2, 53% GF3 and 10% GF4.

According to a second embodiment of the invention, it may be a mixture of oligosaccharides and/or polysaccharides of different types. The invention relates in particular to the use of a mixture of inulin with a GOS, an FOS, oligosaccharides derived from soya, pyrodextrins, an isomaltooligosachharide, a xylooligosaccharide and/or a transgalactooligosaccharide. According to one particular embodiment, a mixture of inulin and a GOS is used, such as Bioline from Gova Ingredients. The invention also relates to the use of a mixture of a GOS with an FOS.

Preferably, in this second mode, the composition according to the invention comprises a mixture of oligosaccharides comprising:
at least one glucooligosaccharide (GOS), and
at least one fructooligosaccharide (FOS),

wherein said glucooligosaccharide (GOS) is present in the composition according to the invention in a concentration of between 0.01% and 10% by weight, preferably between 0.05% and 5% by weight, even better still between 0.1% and 1%, even more preferably between 0.2% and 0.8% by weight relative to the total weight of the composition;

wherein said fructooligosaccharide (FOS) is present in the composition according to the invention in a concentration of between 0.001% and 5% by weight, preferably between 0.01% and 1% by weight, even better still between 0.01% and 0.5%, even more preferably between 0.05% and 0.3% by weight relative to the total weight of the composition.

Advantageously, in the second embodiment of the invention, the composition according to the invention comprises a mixture of oligosaccharides comprising:
at least one glucooligosaccharide (GOS), and
at least one fructooligosaccharide (FOS),

in which the [GOS/FOS] mass ratio is at least 2, preferably is between 2 and 4, even better still between 3 and 4.

Use may in particular be made of the mixture of prebiotic oligosaccharides and probiotic microorganisms sold under the name Ecoskin® by Solabia.

Said mixture comprises in particular:
between 60% and 80% by weight of at least one glucooligosaccharide (GOS) relative to the total weight of the mixture, and
between 10% and 25% by weight of at least one fructooligosaccharide (FOS) relative to the total weight of the mixture.

In particular, said mixture comprises 70% by weight of glucooligosaccharide (GOS), 19% by weight of Polymnia sonchifolia tuber juice, 1% by weight of Lactobacillus acidophilus and Lactobacillus casei, 10% by weight of maltodextrin.

Probiotics

Independently of the abovementioned two variants, the composition according to the invention can also comprise at least one probiotic microorganism.

For the purposes of the present invention, the term “probiotic microorganism” is intended to mean a live microorganism which, when consumed in adequate amount, has a positive effect on the health of its host (“Joint FAO/WHO Expert Consultation on Evaluation of Health and Nutritional Properties of Probiotic in Food Including Powder Milk with Live Lactic Acid Bacteria, 6 Oct. 2001”), and which may in particular improve the intestinal microbial equilibrium.

In the case of the skin, said probiotic microorganism is a probiotic microorganism which, when applied to the skin in an appropriate amount, has a positive effect on the aesthetic qualities of the skin and mucous membranes.

More particularly, they are probiotic microorganisms from the group of lactic acid bacteria, such as in particular Lactobacillus. By way of illustration of these lactic acid bacteria, mention may more particularly be made of Lactobacillus casei, Lactobacillus acidophilus and mixtures thereof.

Specific examples of probiotic microorganisms are Lactobacillus acidophilus, Lactobacillus alimentarius, Lactobacillus curvatus, Lactobacillus delbruckii subsp. Lactis, Lactobacillus gasseri, Lactobacillus johnsonii, Lactobacillus reuteri, Lactobacillus casei, Lactobacillus rhamnosus (Lactobacillus GG), Lactobacillus sake, Lactococcus lactis, Streptococcus thermophilus, Lactobacillus acidophilus, L. delbrueckii, L. helveticus, L. salivarius, L. curvatus, L. plantarum, L. sakei, L. brevis, L. buchneri, L. fermentum, L. reuteri, Lactobacillus bulgaricus, Lactobacillus longum, Lactobacillus lactis, Bifidobacterium longum and mixtures thereof.

The microorganism(s) may be included in the composition according to the invention in a live, semi-active or inactivated, dead form.

They may also be included in the form of fractions of cell components or in the form of metabolites. The microorganism(s), metabolite(s) or fraction(s) may also be introduced in the form of a lyophilized powder, a culture supernatant and/or, where appropriate, in a concentrated form.

According to one preferred embodiment of the invention, these microorganisms are in inactivated form.

The terms “in inactivated form”, “in non-revivable form” and “in dead form” are synonymous herein.

Bacteria “in semi-active form” are bacteria which have partially or totally lost their possibly pathogenic properties.

In general, the compositions according to the invention generally comprise from 0.0001 to 20% by weight of at least one probiotic microorganism relative to the total weight of the composition.

Advantageously, the probiotic microorganism is present in the composition according to the invention in a concentration of between 0.0001% and 10% by weight, preferably between 0.001% and 5% by weight, even more preferentially between 0.001% and 1% by weight relative to the total weight of the composition.

This or these microorganism(s) can be included in the compositions according to the invention in a live, semi-active or inactivated, dead form, preferably inactivated for example by heat or by high pressure.

In the case where the microorganisms are formulated in a composition in a live form, the amount of live microorganisms may range from 10³ to 10¹⁵ cfu/g, in particular from 10⁵ to 10¹⁵ cfu/g and more particularly from 10⁷ to 10¹² cfu/g of microorganisms per gram of composition.

In one particularly preferred embodiment, the composition according to the invention comprises a mixture of oligosaccharides comprising:
at least one glucooligosaccharide (GOS), and
at least one fructooligosaccharide (FOS),

wherein said glucooligosaccharide (GOS) is present in the composition according to the invention in a concentration of between 0.01% and 10% by weight, preferably between 0.05% and 5% by weight, even better still between 0.1% and 1%, even more preferably between 0.2% and 0.8% by weight relative to the total weight of the composition;

wherein said fructooligosaccharide (FOS) is present in the composition according to the invention in a concentration of between 0.001% and 5% by weight, preferably between 0.01% and 1% by weight, even better still between 0.01% and 0.5%, even more preferably between 0.05% and 0.3% by weight relative to the total weight of the composition;

and in addition at least one probiotic microorganism chosen from bacteria of the Lactobacillus genus, in particular Lactobacillus casei, Lactobacillus acidophilus and mixtures thereof, wherein said probiotic microorganism is between 0.0001% and 10% by weight, preferably between 0.001% and 5% by weight, even better still between 0.001% and 1% by weight relative to the total weight of the composition.

Advantageously, the composition according to the invention comprises a mixture of oligosaccharides comprising:
at least one glucooligosaccharide (GOS), and
at least one fructooligosaccharide (FOS),

in which the [GOS/FOS] mass ratio is at least 2, preferably is between 2 and 4, even better still between 3 and 4.

Use may in particular be made of the mixture of prebiotic oligosaccharides and probiotic microorganisms sold under the name Ecoskin® by Solabia.

Said mixture comprises in particular:
between 60% and 80% by weight of at least one glucooligosaccharide (GOS) relative to the total weight of the mixture, and
between 10% and 25% by weight of at least one fructooligosaccharide (FOS) relative to the total weight of the mixture, and
between 0.001 and 15% by weight of at least one probiotic microorganism, in particular chosen from Lactobacillus casei, Lactobacillus acidophilus and mixtures thereof, relative to the total weight of the mixture.

In particular, said mixture comprises 70% by weight of glucooligosaccharide (GOS), 19% by weight of Polymnia sonchifolia tuber juice, 1% by weight of Lactobacillus acidophilus and Lactobacillus casei, 10% by weight of maltodextrin.

As mentioned previously, the composition according to the invention comprises a physiologically acceptable medium. More particularly, said physiologically acceptable medium may comprise water and/or one or more water-soluble organic solvents which can be chosen from linear or branched C₁-C₆ monoalcohols such as ethanol, isopropanol, tert-butanol or n-butanol; polyols such as glycerol, propylene glycol, hexylene glycol (or 2-methyl-2,4-pentanediol), and polyethylene glycols; polyol ethers such as dipropylene glycol monomethyl ether; and mixtures thereof.

Preferentially, the composition according to the invention has a water content ranging from 20% to 95% by weight, even better still from 40% to 90% by weight relative to the total weight of the composition.

Advantageously, the composition comprises one or more water-soluble organic solvents in a content ranging from 0.5% to 25% by weight, preferably from 5% to 20% by weight, even better still from 10% to 15% by weight relative to the total weight of the composition.

Presentation Forms

A cosmetic composition according to the invention is in particular applied topically.

The compositions intended for external topical administration may be aqueous, aqueous-alcoholic or oily solutions, solutions or dispersions of the lotion or serum type, emulsions of liquid or semi-liquid consistency of the milk type, obtained by dispersion of a fatty phase in an aqueous phase (O/W) or vice versa (W/O), or suspensions or emulsions, of soft, semi-solid or solid consistency, of the cream type, aqueous or anhydrous gels, microemulsions, microcapsules, microparticles, or vesicular dispersions of ionic and/or non-ionic type.

These compositions are prepared according to the usual methods.

A topical composition according to the invention can advantageously be formulated in any presentation form suitable for skin care, in particular in the form of creams for cleansing, protecting, treating or caring for the face, or for the body, of a mask to be left placed on the skin or the hair, of makeup-removal milks, of body protection or care milks, of lotions, gels or foams for skin care, such as cleansing lotions, bath compositions.

Alternatively, a topical composition according to the invention can advantageously be formulated in any presentation form suitable for hair care, in particular in the form of a hair lotion, of a shampoo, in particular an anti-dandruff shampoo, of a conditioner, of a disentangler, a hair cream or gel, of a treating lotion, of a lotion or gel for preventing hair loss, of an anti-parasitic shampoo, or of a treating shampoo, in particular an anti-seborrhoeic shampoo, of a scalp care, in particular anti-irritant, anti-ageing, restructuring, product.

Adjuvants

In a known manner, galenical forms intended for topical administration may also contain adjuvants that are common in the cosmetic, pharmaceutical and/or dermatological field, such as hydrophilic or lipophilic gelling agents, hydrophilic or lipophilic active agents, preservatives, antioxidants, solvents, fragrances, fillers, screening agents, fatty substances such as oils, emulsifiers, odour absorbers and colourants. The amounts of these various adjuvants are those conventionally used in the field under consideration, for example from 0.01% to 20% of the total weight of the composition. Depending on their nature, these adjuvants may be introduced into the fatty phase and/or into the aqueous phase.

As hydrophilic gelling agents that can be used in the composition according to the invention, mention may be made of modified or unmodified carboxyvinyl polymers, such as the products sold under the names Carbopol (INCI name: carbomer) and Pemulen (INCI name: Acrylates/C10-30 alkyl acrylate crosspolymer) by the company Goodrich, or such as the crosslinked sodium polyacrylate sold under the name Cosmedia SP by the company Cognis (INCI name: Sodium polyacrylate); polyacrylamides; 2-acrylamido-2-methylpropanesulfonic acid homopolymers such as the product sold by Clariant under the name Hostacerin AMPS (INCI name: ammonium polyacryldimethyltaurate); crosslinked anionic copolymers of acrylamide and AMPS, provided in the form of an emulsion, such as those sold under the name of Sepigel 305 (name CTFA: Polyacrylamide/C13-14 isoparaffin/Laureth-7) and under the name Simulgel 600 (CTFA name: Acrylamide/Sodium acryloyldimethyltaurate copolymer/Isohexadecane/Polysorbate 80) by the company SEPPIC; acrylate/acrylonitrile copolymers such as Hypan SS201 sold by the company Kingston; synthetic neutral polymers such as poly-N-vinylpyrrolidone; polysaccharides such as guar gum, xanthan gum and cellulose-based derivatives. The amount of these polymers can range, for example, from 0.05 to 5% by weight and better still from 0.1 to 3% by weight relative to the total weight of the composition.

Lipophilic gelling agents that may be mentioned include modified clays, such as bentones, metal salts of fatty acids, such as aluminium stearates, and hydrophobic silica, or alternatively ethylcellulose and polyethylene.

When a composition of the invention is an emulsion, the proportion of the fatty phase may range from 5% to 80% by weight and preferably from 10% to 50% by weight relative to the total weight of the composition. The oils, emulsifiers and coemulsifiers used in the composition in emulsion form are chosen from those conventionally used in cosmetics and/or dermatology. The emulsifier and the coemulsifier may be present in the composition in a proportion ranging from 0.3% to 30% by weight and preferably from 0.5% to 20% by weight relative to the total weight of the composition.

When the composition of the invention is an oily solution or gel, the fatty phase may represent more than 90% of the total weight of the composition.

As oils that may be used, examples that may be mentioned include:
hydrocarbon-based oils of plant origin, such as liquid fatty acid triglycerides including from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stéarineries Dubois or those sold under the names Miglyol 810, 812 and 818 by the company Dynamit Nobel, jojoba oil and shea butter oil;
synthetic esters and ethers, especially of fatty acids, for instance the oils of formulae R₁COOR₂ and R₁OR₂ in which R₁ represents a fatty acid residue containing from 8 to 29 carbon atoms and R₂ represents a branched or unbranched hydrocarbon-based chain containing from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate, and fatty alcohol heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;
linear or branched hydrocarbons, of mineral or synthetic origin, such as paraffin oils, which may be volatile or non-volatile, and derivatives thereof, petroleum jelly, polydecenes, hydrogenated polyisobutene such as parleam oil, fatty alcohols having from 8 to 26 carbon atoms, such as cetyl alcohol, stearyl alcohol and the mixture thereof (cetylstearyl alcohol), octyldodecanol, 2-butyloctanol, 2-hexyldecanol, 2-undecylpentadecanol, oleyl alcohol or linoleyl alcohol; volatile or non-volatile silicone oils such as polymethylsiloxanes (PDMSs) with a linear or cyclic silicone chain, which are liquid or pasty at ambient temperature, in particular cyclopolydimethylsiloxanes (cyclomethicones) such as cyclohexasiloxane; polydimethylsiloxanes comprising alkyl, alkoxy or phenyl groups, which are pendent or at the end of the silicone chain, said groups having from 2 to 24 carbon atoms; phenylated silicones such as phenyl trimethicones, phenyl dimethicones, phenyltrimethylsiloxydiphenylsiloxanes, diphenyl dimethicones, diphenylmethyldiphenyltrisiloxanes, 2-phenylethyltrimethylsiloxysilicates, and polymethylphenylsiloxanes; and
mixtures thereof.

In the list of oils mentioned above, the term “hydrocarbon-based oil” means any oil predominantly including carbon and hydrogen atoms, and possibly ester, ether, fluoro, carboxylic acid and/or alcohol groups.

As emulsifiers, mention may be made of amphoteric, cationic, anionic or non-ionic surfactants, used alone or as a mixture, and optionally a co-emulsifier.

For O/W emulsions, mention may for example be made, as emulsifiers, of non-ionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters, in particular oxyalkylenated sugar esters, esters of phosphoric acid and of a fatty alcohol; and mixtures thereof.

In one preferred embodiment, the composition according to the invention also comprises at least one ingredient chosen from silicone fatty substances such as silicone oils, gums and waxes; non-silicone fatty substances such as oils, pastes and waxes of plant, mineral, animal and/or synthetic origin; fatty acids having from 8 to 32 carbon atoms; synthetic esters and ethers, in particular of formula R¹COOR² and R¹OR² in which R¹ represents the residue of a fatty acid comprising from 8 to 29 carbon atoms, and R² represents a branched or unbranched hydrocarbon-based chain containing from 3 to 30 carbon atoms; linear or branched hydrocarbons of mineral or synthetic origin; fatty alcohols having from 8 to 26 carbon atoms; water; C₂-C₆ monoalcohols; glycols chosen from propylene glycol, butylene glycol, pentylene glycol; ketones; thickeners, emulsifiers, surfactants, gelling agents, fragrances, fillers, colourants, moisturizers, vitamins chosen from vitamin A, E, B3, polymers.

The amounts of these various ingredients are those conventionally used in the field under consideration, for example from 0.01% to 20% of the total weight of the composition.

Cosmetic Treatment Processes and Cosmetic Uses

The present invention also relates to a cosmetic treatment process for skin care comprising the application to the skin of the composition according to the invention.

In particular, the treatment process aims to prevent and/or reduce the effects of oxidative stress or free radicals, in particular the effects of UV rays, on keratin materials, in particular the skin.

It also relates to a cosmetic treatment process for the skin intended to prevent and/or treat the signs of skin ageing, in particular induced by oxidative stress.

The term “signs of skin ageing” is intended to mean any modification of the external appearance of the skin due to ageing of chronological and/or photo-induced origin.

The term “oxidative stress” as used in the present application cover all of the damage caused by an increase in oxygen free radicals in a subject.

Among the signs of skin ageing induced in particular by oxidative stress, mention may be made of a surface that is not very homogeneous and is less smooth surface, a thinned epidermis, wrinkles and fine lines, dry skin, a lack of elasticity and/or of tone of the skin, resulting in the appearance of flaccid, wrinkled skin.

In particular, the signs of skin ageing targeted by the invention are chosen from a thinning of the skin, a loss of firmness, a loss of elasticity, a loss of density or a loss of skin tone, an alteration of the surface of the skin, the appearance of a marked microrelief of the skin, the appearance of roughness, the formation and/or the presence of fine lines and/or wrinkles, a change in the radiance of the skin complexion, a tanned appearance of the skin, sagging skin or wizened skin.

Preferably, the signs of skin ageing targeted by the invention are chosen from thinning of the skin, the appearance of a marked microrelief of the skin, the formation and/or the presence of fine lines and/or wrinkles, sagging skin and wizened skin.

Preferentially, the signs of skin ageing targeted by the invention are chosen from the appearance of a marked microrelief of the skin, the formation and/the presence of fine lines and/or wrinkles, sagging skin and dry skin.

The present invention also relates to the cosmetic use of the composition according to the invention for preventing and/or treating the signs of skin ageing, in particular induced by oxidative stress.

A subject of the invention is also the cosmetic use of the composition according to the invention for protecting the skin against oxidative stress.

### FIGURES

FIG. 1 Time course of C. acnes bacterial growth (optical density OD as a function of time) in the presence of the preparations of starting materials tested alone.

FIG. 2 Time course of C. acnes bacterial growth (optical density OD as a function of time) in the presence of the preparations of starting materials tested as a mixture.

The examples that follow illustrate the invention, and are given purely as nonlimiting illustrations.

Throughout the text which follows, the percentages are given on a weight basis, unless otherwise mentioned.

The expression “at least one” is equivalent to “one or more”.

The expressions “between . . . and . . . ” and “ranging from . . . to . . . ”, “at least . . . ” or “at most” should be understood as being limits inclusive, unless otherwise specified.

The examples and figures that follow are provided as illustrations which do not limit the field of the invention.

### EXAMPLES

### Example 1—Comparison of the Effect of Combinations of Bifidobacterium longum Lysate+Saccharomyces cerevisiae Yeast Extract+Mannose and Bifidobacterium longum Lysate+Saccharomyces cerevisiae Yeast Extract+Mannose+Oligosaccharides+Lactobacillus on the Growth of Cutibacterium acnes Compared to Each of the Starting Materials Alone

C. acnes ATCC 6919 is stored at −80° C. Nine days before the test, the strain is placed in a TSA (Tryptone Soy Agar) plate. Four days before the test, an inoculum of 10 ml of TSB (tryptone soy broth) at an OD620 nm of 0.07 is produced, then this inoculum is diluted 1/10 and then incubated at 35° C. On the day of the test, a preparation of a suspension at OD620 nm=0.07 is prepared. 4.5 ml of the preparations of starting materials alone or in combination are added to 0.5 ml of this bacterial suspension.

This step is the “time 0 (T0)” of the time course. The cultures are incubated at (35° C.) under anaerobic conditions.

A point is taken every day starting from 72 hours: T0, T72 h, T96 h, T120 h.

The preparations of starting materials (SM) containing the starting material to be tested alone or as a mixture, TSB ½ and Versol water as diluent.

TSB diluted 50/50 (w/w) with sterile Versol water (TSB ½) is used as the minimum medium promoting minimal growth making it possible to detect the added nutritional value of each SM tested.

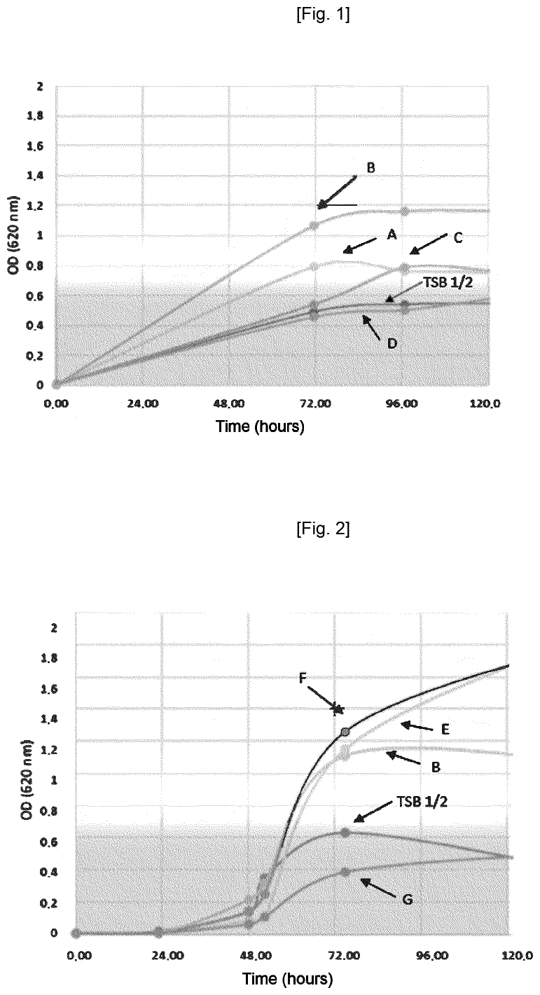

The graphs of FIGS. 1 and 2 show the results obtained.

The higher the optical density measured, the greater the growth of C. acnes.

Starting Materials Tested Alone

TABLES 1

| Strain tested | Cutibacterium acnes ATCC 6919 |
|---|---|
| Bacterial | A: 0.5 ml of C. acnes suspension and 4.5 ml of ½ TSB + 10% |
| cultures of | Bifidobacterium longum lysate (Repair Complex CLR ® sold by the |
| which the OD | company K. Richter GmbH) |
| is measured | B: 0.5 ml of C. acnes suspension + 4.5 ml of ½ TSB + 1% of |
| | Saccharomyces cerevisiae yeast extract (Firmalift ® GR sold by |
| | the company Silab) |
| | C: 0.5 ml of C. acnes suspension + 4.5 ml of ½ TSB + 0.5% of D- |
| | mannose sold by the company Danisco Sweeteners |
| | D: 0.5 ml of C. acnes suspension + 4.5 ml of ½ TSB + 0.3% of a |
| | mixture of 70% of glucooligosaccharide (GOS), 19% of tuber juice |
| | of Polymnia sonchifolia, 1% of Lactobacillus acidophilus and |
| | Lactobacillus casei, 10% maltodextrin (Ecoskin ® sold by Solabia) |

Time course of C. acnes bacterial growth (optical density OD as a function of time) in the presence of the starting materials tested alone—(see FIG. 1)

TABLES 2

| Time | 0.00 | 71.50 | 96.75 | 120.75 | 120.75 |
|---|---|---|---|---|---|
| (hours) | | | | | |
| Optical density (OD) | | | | | OD_{(culture)} − OD_{(control)} |
| TSB ½ | 0.004 | 0.49 | 0.54 | 0.548 | 0 |
| Control | | | | | |
| A | 0.004 | 0.791 | 0.763 | 0.755 | 0.207 |
| B | 0.002 | 1.065 | 1.161 | 1.165 | 0.617 |
| C | 0.008 | 0.54 | 0.788 | 0.768 | 0.22 |
| D | 0.009 | 0.455 | 0.501 | 0.579 | 0.031 |

Conclusion: The results of Table 2 show that the SM present in culture B exhibits a strong effect on the growth of C. acnes compared to the TSB ½ alone. The SMs present in cultures A and C both have a moderate effect on the growth of C. acnes compared to the TSB ½. Finally, it is noted that the SM present in culture D has no effect on the growth of C. acnes because its growth is identical to that of the TSB ½ alone.

Mixtures of Starting Materials Tested

TABLES 3

| Strain tested | Cutibacterium acnes ATCC 6919 |
|---|---|
| Bacterial cultures | E: 0.5 ml of C. acnes suspension + 4.5 ml of ½ TSB + 10% |
| of which the OD is | Bifidobacterium longum lysate (Repair Complex CLR ® sold |
| measured | by the company K. Richter GmbH) + 1% Saccharomyces |
| | cerevisiae yeast extract (Firmalift ® GR sold by the company |
| | Silab) + 0.5% of D-mannose sold by the company Danisco |
| | Sweeteners + 0.3% of a mixture of 70% of |
| | glucooligosaccharide (GOS), 19% of Polymnia sonchifolia |
| | tuber juice, 1% of Lactobacillus acidophilus and Lactobacillus |
| | casei, 10% of maltodextrin (Ecoskin ® sold by the company |
| | Solabia) |
| | F: 0.5 ml of C. acnes suspension + 4.5 ml of ½ TSB + 10% |
| | Bifidobacterium longum lysate (Repair Complex CLR ® sold |
| | by K. Richter GmbH) + 1% Saccharomyces cerevisiae yeast |
| | extract (Firmalift ® GR sold by the company Silab) + 0.5% of |
| | D-mannose sold by the company Danisco Sweeteners |
| | G: 0.5 ml of C. acnes suspension + 4.5 ml of ½ TSB + 0.5% |
| | of D-mannose sold by the company Danisco Sweeteners + |
| | 0.3% of a mixture of 70% of glucooligosaccharide (GOS), |
| | 19% of tuber juice of Polymnia sonchifolia, 1% of |
| | Lactobacillus acidophilus and Lactobacillus casei, 10% |
| | maltodextrin (Ecoskin ® sold by Solabia) |

Time course of C. acnes bacterial growth (optical density OD as a function of time) in the presence of the starting materials tested as a mixture—(see FIG. 2)

TABLE 4

| | Time (hours) | | | | | | |
|---|---|---|---|---|---|---|---|
| | | | | | | | 143.67 |
| | 0.00 | 23.00 | 48.08 | 52.50 | 74.83 | 143.67 | OD_{(culture)} − |
| Optical density (OD) | | | | | | | OD_{(control)} |
| TSB ½ | 0.006 | 0.019 | 0.143 | 0.35 | 0.631 | 0.378 | 0 |
| Control | | | | | | | |
| B | 0.004 | 0.006 | 0.213 | 0.318 | 1.109 | 1.069 | 0.691 |
| E | 0 | 0.003 | 0.083 | 0.111 | 1.148 | 1.872 | 1.494 |
| F | 0.003 | 0.005 | 0.141 | 0.248 | 1.258 | 1.825 | 1.447 |
| G | 0.007 | 0.008 | 0.061 | 0.107 | 0.386 | 0.513 | 0.135 |

Conclusion: The results of Table 4 show that the mixtures of SM present in cultures E and F both have a strong effect on the growth of C. acnes to ½ TSB and also compared to the SM present in culture B. Finally, it is noted that the mixture of SM present in culture G has no effect on the growth of C. acnes because its growth is very slightly higher than that of ½ TSB alone.

OD of cultures A+B+C+D=1.075<OD of culture E=1.494. Thus, the mixture of SM present in culture E exhibits a synergistic effect on the growth of C. acnes compared to the sum of the effects of the SMs alone present in cultures A, B, C and D (1.075). Furthermore, the mixture of SM present in culture E has a greater effect on the growth of C. acnes compared to the SM present in culture B.

OD of cultures A+B+C=1.044<OD of culture F=1.447. Thus, the mixture of SM present in culture F exhibits a synergistic effect on the growth of C. acnes compared to the sum of the effects of the SMs alone present in cultures A, B and C (1.044). Furthermore, the mixture of SM present in culture F has a greater effect on the growth of C. acnes compared to the SM present in culture B.

### Example 2—Cosmetic Composition According to the Invention

Composition No. 1 was prepared according to the following invention:

TABLES 5

| | Composition |
|---|---|
| | No. 1 |
| | according to |
| | the invention |
| INCI names | (m/m) |
| BIFIDOBACTERIUM LONGUM LYSATE⁽¹⁾ | 10 |
| 96% ETHANOL | 5 |
| MANNOSE⁽²⁾ | 0.5 |
| ACTIVE AGENTS | 1.765 |
| SODIUM HYDROXIDE | 0.0345 |
| ALPHA-GLUCAN OLIGOSACCHARIDE (and) | 0.3 |
| POLYMNIA SONCHIFOLIA tuber juice (and) | |
| LACTOBACILLUS⁽³⁾ | |
| SACCHAROMYCES CEREVISIAE YEAST | 1 |
| EXTRACT⁽⁴⁾ | |
| OIL | 1.5 |
| FRAGRANCE | qs |
| GELLING AGENT | 0.35 |
| OXYETHYLENATED (60 EO) HYDROGENATED | 0.0165 |
| CASTOR OIL | |
| PEG-20 METHYL GLUCOSE SESQUISTEARATE | 0.1 |
| DISODIUM EDTA | 0.1 |
| GLYCOL | 5 |
| PRESERVATIVES | qs |
| WATER | qs 100 |

⁽¹⁾Repair Complex CLR ® sold by the company K. Richter GmbH.
⁽²⁾D-Mannose, sold by the company Danisco.
⁽³⁾Ecoskin ® sold by the company Solabia comprising 70% by weight of glucooligosaccharide (GOS), 19% by weight of Polymnia sonchifolia tuber juice, 1% by weight of Lactobacillus acidophilus and Lactobacillus casei, 10% by weight maltodextrin.
⁽⁴⁾Firmalift ® GR sold by the company Silab comprising a Saccharomyces cerevisiae yeast extract at a concentration of 4.2% in an aqueous-glycolic solution comprising 7.5% of pentylene glycol and 88.3% of water.

Composition No. 1 was prepared as follows:

The compounds that cannot be dissolved cold are mixed and heated to 80° C. in order to form a pre-emulsion. This is then cooled to 35° C. The gelling agents are then added and dissolved. The probiotic fractions are added at ambient temperature.

The anti-ageing composition No. 1 was applied to the skin of the face.

1. A cosmetic composition comprising, in a physiologically acceptable medium:
at least one microorganism of the Bifidobacterium species genus and/or a fraction thereof and/or a metabolite thereof, and
at least one extract of yeast of the Saccharomyces genus, and
at least one monosaccharide chosen from mannose, rhamnose, and mixtures thereof,
said microorganism being used in the form of a lysate.

2. The composition according to claim 1, in which the microorganism of the Bifidobacterium species genus is chosen from Bifidobacterium longum, Bifidobacterium bifidum, Bifidobacterium breve, Bifidobacterium animalis, Bifidobacterium lactis, Bifidobacterium infantis, Bifidobacterium adolescentis or Bifidobacterium pseudobacterium and mixtures thereof.

3. The composition according to claim 1, in which the microorganism of the Bifidobacterium species genus is Bifidobacterium longum.

4. The composition according to claim 1, in which said lysate is present in a concentration of between 0.001% and 20% by dry weight relative to the total weight of the composition.

5. The composition according to claim 1, in which said yeast extract is present in a concentration of between 0.001% and 5% by weight relative to the total weight of the composition.

6. The composition according to claim 1, in which said monosaccharide is present in a concentration of between 0.01% and 20% by weight relative to the total weight of the composition.

7. The composition according to claim 1, also comprising at least one oligosaccharide and/or polysaccharide chosen in the group constituted of inulin, a fructooligosaccharide, a glucooligosaccharide, oligosaccharides derived from soya beans, pyrodextrins, an isomaltooligosaccharide, a xylooligosaccharide, a transgalactooligosaccharide and mixtures thereof.

8. The composition according to claim 7, in which said oligosaccharide and/or polysaccharide is present in a concentration of between 0.01% and 20% by weight relative to the total weight of the composition.

9. The composition according to claim 7, in which said oligosaccharide and/or polysaccharide is in the form of a mixture comprising:
at least one glucooligosaccharide (GOS), and
at least one fructooligosaccharide (FOS),
wherein said glucooligosaccharide (GOS) is present in the composition according to the invention in a concentration of between 0.01% and 10% by weight relative to the total weight of the composition;
wherein said fructooligosaccharide (FOS) is present in the composition according to the invention in a concentration of between 0.001% and 5% by weight relative to the total weight of the composition.

10. The composition according to claim 1, also comprising at least one probiotic microorganism.

11. The composition according to claim 10, in which said probiotic microorganism is present in a concentration of between 0.0001% and 10% by weight relative to the total weight of the composition.

12. A cosmetic treatment process for skincare comprising the application to the skin of the composition as defined in claim 1.

13. The process according to claim 12, for preventing and/or treating the signs of skin ageing.

14. The process according to claim 13, for protecting the skin against oxidative stress.

15. (canceled)

16. (canceled)

17. (canceled)

18. The composition according to claim 1, in which the lysate is present in a concentration of between 0.001% and 2% by dry weight relative to the total weight of the composition.

19. The composition according to claim 1, in which the monosaccharide is present in a concentration of from 0.1% to 5% by weight relative to the total weight of the composition.

20. The composition according to claim 1, in which the monosaccharide is present in a concentration of from 0.05% to 10% by weight relative to the total weight of the composition.

21. The composition according to claim 7, in which the oligosaccharide and/or polysaccharide is present in a concentration of between 0.05% and 10% by weight relative to the total weight of the composition.

22. The composition according to claim 7, in which said oligosaccharide and/or polysaccharide is in the form of a mixture comprising:
at least one glucooligosaccharide (GOS), and
at least one fructooligosaccharide (FOS),
wherein the glucooligosaccharide (GOS) is present in the composition according to the invention in a concentration of between 0.05% and 5% by weight relative to the total weight of the composition;
wherein the fructooligosaccharide (FOS) is present in the composition according to the invention in a concentration of between 0.01% and 1% by weight relative to the total weight of the composition.

23. The composition according to claim 7, in which the oligosaccharide and/or polysaccharide is in the form of a mixture comprising:
at least one glucooligosaccharide (GOS), and
at least one fructooligosaccharide (FOS),
wherein the glucooligosaccharide (GOS) is present in the composition according to the invention in a concentration of between 0.1% and 1% by weight relative to the total weight of the composition;
wherein the fructooligosaccharide (FOS) is present in the composition according to the invention in a concentration of between 0.01% and 0.5% by weight relative to the total weight of the composition.
